(19) United States
(12) Patent Application Publication
(10) Pub. No.: US 2020/0043605 A1
(43) Pub. Date: Feb. 6, 2020

(54) SURGICAL INSTRUMENT MANAGEMENT SYSTEM

(71) Applicant: DGSHAPE Corporation, Hamamatsu-shi (JP)

(72) Inventor: Masaki HANAJIMA, Hamamatsu-shi (JP)

(21) Appl. No.: 16/531,170

(22) Filed: Aug. 5, 2019

(30) Foreign Application Priority Data
Aug. 6, 2018 (JP) 2018-147678

(51) Int. Cl. G16H 40/40; A61B 34/00; A61B 90/90

(52) U.S. Cl. CPC G16H 40/40; A61B 34/25; G16H 40/60; A61B 2034/258; A61B 2034/256; A61B 90/90

(57) ABSTRACT

A surgical instrument management system includes a first memory, a second memory, a third memory, and a first processor. The first memory stores user IDs associated in a one-to-one correspondence with users. The second memory stores instrument IDs associated in a one-to-one correspondence with surgical instruments. The third memory stores the instrument IDs, work information and work hours of work performed on the instruments each identified by a respective one of the instrument IDs, and the user IDs so that the instrument IDs, the work information, the work hours and the user IDs are associated with each other. The first processor extracts work information of one of the users identified by a corresponding one of the user IDs in a predetermined period, based on the user IDs and the work hours stored in the third memory.

### CROSS REFERENCE TO RELATED APPLICATIONS

This application claims the benefit of priority to Japanese Patent Application No. 2018-147678 filed on Aug. 6, 2018. The entire contents of this application are hereby incorporated herein by reference.

### BACKGROUND OF THE INVENTION

### 1. Field of the Invention

The present invention relates to a management system for surgical instruments.

### 2. Description of the Related Art

JP 2003-016198 A discloses a device that provides identification codes individually for small instruments used in medical consultation, surgical operation, and medical examination, and that reads the identification codes. Data about patients, data about doctors attending to the patients, inventory data of small steel instruments, and the like are stored as medical data in a database of the managing device. After a medical practice such as a surgical operation and a treatment is carried out for a patient, read data are compared with the medical data stored in the database of the managing device.

JP 2009-72338 A discloses a management system for a medical device that is cleaned after every one use for repeated use. JP 2009-72338 discloses that a display part displays different colors according to the state of an endoscope.

JP 2012-215990 A discloses a device for supporting a picking work of medical instruments and materials. Specifically, the device includes a storage part that stores identifiers of medical instruments and materials and images of the medical instruments and materials, and another storage part that associates identifiers of mutually similar medical instruments and materials with each other and stores them. The device disclosed in JP 2012-215990 A is configured to be able to display an image of a medical instrument or material specified by the user and an image of medical instrument or material that is similar the specified medical instrument or material side by side.

JP 2013-116234 A discloses an apparatus that manages the locations of medical devices. The apparatus disclosed in JP 2013-116234 A has a device information acquisition part that acquires device information including read information of identification tags attached to the medical devices from tag readers installed at a plurality of locations in a medical facility. A status holding part holds the statuses of the medical devices, which includes the locations of the medical devices that are identified by the acquired device information. A display control part causes a display device to display the statuses of the medical devices held by the status holding part. The display control part also causes the display device to hide the medical device in use for a medical practice among the medical devices.

JP 2015-197735 A discloses a device capable of distributing guidance information that helps general medical staff. The device disclosed in JP 2015-197735 A uses medical work information that represents details of medical practices carried out by experienced medical staff as the guidance information. Then, the device distributes the guidance information in response to a request from general medical staff.

Japanese Registered Utility Model No. 3199614 discloses a surgical instrument management system. The system disclosed in Japanese Registered Utility Model No. 3199614 records photographs of external appearance related to surgical instruments. The system uses bar code information to create an asset number list of the surgical instruments prepared before a surgical operation. Next, the system reads identification patterns of the surgical instruments that were collected after the surgical operation. The system displays the external appearance photographs of the surgical instruments, and also determines whether or not the asset numbers of the surgical instruments collected after the surgical operation match the asset numbers of the surgical instruments prepared before the surgical operation.

### SUMMARY OF THE INVENTION

Generally, surgical instruments are managed after a surgical operation in the following sequence; collection, cleaning, assembling, sealing, sterilization, and storage. The instruments are sterilized and thereafter stored, and used again in another surgical operation. Medical facilities, such as hospitals, possess many types of instruments and surgical instrument sets, and they store a plurality of instruments and surgical instrument sets of each type. When in use, the stored instruments or surgical instrument sets are picked out each time. In the field of surgical instrument management, a system has not yet been proposed that can provide data to assess the productivity and the proficiency level of workers.

A surgical instrument management system proposed in this disclosure includes, for example, a first memory, a second memory, a third memory, and a first processor. The first memory is provided to store user IDs associated in a one-to-one correspondence with users. The second memory is provided to store instrument IDs associated in a one-to-one correspondence with surgical instruments. The third memory is provided to store the user IDs, the instrument IDs, and work information and work hours of work performed on the instruments each identified by a respective one of the instrument IDs so that the instrument IDs, the work information, the work hours, and the user IDs are associated with each other. The first processor is configured or programmed to extract work information of one of the users identified by a corresponding one of the user IDs in a predetermined period Z1, based on the user IDs and the work hours stored in the third memory.

Preferred embodiments of the surgical instrument management systems are able to extract work information of users in a predetermined period Z1. Based on the obtained information, it is possible to provide data to assess the productivity and the proficiency level of workers, for example.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a schematic view illustrating a surgical instrument management system 10.

FIG. 2 is a schematic view illustrating an example of an application of the system 10.

FIG. 3 is a view illustrating an example of the configuration of a database defining and functioning as a first memory 101 that stores user IDs.

FIG. 4 is a view illustrating an example of the configuration of a database 400 in which a second memory 102 and a fourth memory 104 are integrated into a single database.

FIG. 5 is a view illustrating a table 420 that records work information for a collection area A2.

FIG. 6 is a view illustrating a table 440 that records work information for a sterilization area A5.

FIG. 7 is a view illustrating a table 460 that records work information for a repair area A7.

FIG. 8 is a view illustrating a dashboard provided for this system 10.

FIG. 9 is a view illustrating an example of the configuration of a pre-stored master data table that stores outsourced contractor data, outsourcing work information, and outsourcing unit prices, which are associated with each other.

FIG. 10 is a view illustrating an example of an information display screen containing a graph showing the work effort hours per unit time in graphical representation.

### DETAILED DESCRIPTION OF THE PREFERRED EMBODIMENTS

Hereinbelow, surgical instrument management systems according to preferred embodiments of the present invention will be described with reference to the drawings. It should be noted, however, that the preferred embodiments described herein are, of course, not intended to limit the present invention.

Herein, a surgical instrument set refers to a set of a plurality of medical instruments that are used in a surgical operation or in one of the steps of the surgical operation. Herein, the medical instruments may be referred to simply as “instruments”, as appropriate.

Examples of the instruments of the surgical instrument set include trocars, forceps, cutting devices, cleaning and suction devices, scissors (Cooper scissors), scalpels, scalpel holders, cannulae, thumb forceps, retractors, scales, sounds, elevators, raspatories, suction tubes, rib spreaders, rib contractors, needle holders, injection syringes, metal basins, pus basins, glasses, pins, mirrors, files, mouth gags, wound clips, handpieces, chisels, sharp spoons, dissectors, surgical needles, intervertebral disc rongeurs, water receivers, needles, depressors, bougies, ventilation tubes, bone impactors, Luer bone rongeurs, needle-nose pliers, hammers, goniometers, pipettes, enemators, and syringes.

The medical instruments may include an instrument including a plurality of component parts. For example, the instruments used in a laparoscopic surgery, such as trocars, forceps, a cutting device, and a cleaning and suction device, are each including a plurality of component parts, and they are collected after the surgical operation and dissembled into a plurality of component parts. Herein, each of the parts that makes up a medical instrument is referred to as a “component part”. Moreover, each component part may further include a plurality of component parts. When this is the case, each of such component parts may also be referred to as a “component part”.

For the medical instruments and the component parts of the medical instruments, different cleaning procedures are specified. For this reason, the medical instruments and the component parts of the instruments are collected after a surgical operation, then sorted by specified cleaning procedures, and then sent to a cleaning step. After the cleaning, the instruments each including a plurality of component parts are assembled. Also, the medical instruments are classified by the type of surgical instrument set or by the type of medical instrument, and they are enclosed and sealed in predetermined containers. Thereafter, the instruments are sterilized in a specified sterilization method, and then stored until they are used next time. Thus, when in use, a surgical instrument set repeatedly undergoes the following cycle of steps: surgical operation→collection (sorting)→cleaning→assembling (sealing)→sterilization→storage→surgical operation. After a surgical operation, surgical instruments and surgical instrument sets undergo the following steps: collection, cleaning, assembling, sealing, sterilization, and storage. For this reason, many hospitals possess many types of surgical instrument sets and surgical instruments, and they store a plurality of surgical instrument sets and surgical instruments. When in use, the stored surgical instrument sets and surgical instruments are picked out each time.

In addition, when some problems are found in surgical instrument sets and surgical instruments, such as blunt blades due to damaged cutting edges, unsmooth engaging movements of forceps, and the like, such surgical instrument sets and surgical instruments may be sent out for repair in some cases. In other cases, such surgical instrument sets and the surgical instruments are sent out for regular inspections, so that necessary processing can be performed. Such repairs and inspections are carried out by specialist contractors outside hospitals. The instruments and the surgical instrument sets that are sent to specialist contractors are out of the normal cycle, which includes a surgical operation, collection, cleaning, sealing, sterilization, and storage.

Thus, the management of surgical instruments involves various kinds of work, including repairs and inspections. This means that work management is complicated and troublesome, and that it is difficult to comprehend how each of the workers is engaged in which work and keep track of the productivity of each of the workers. The surgical instrument management system proposed herein may be used to manage, for example, work and productivity of the workers who manage surgical instruments appropriately and efficiently.

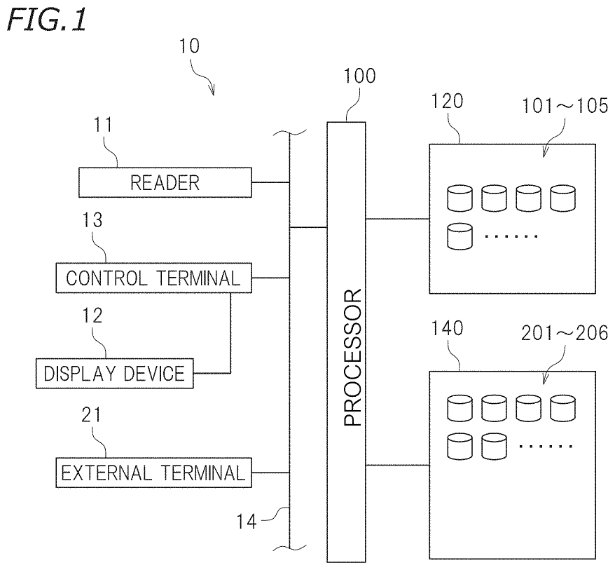

FIG. 1 is a schematic view of a surgical instrument management system 10 (which may be simply referred to as a “system 10” when appropriate in this description). In the preferred embodiment shown in FIG. 1, the system 10 includes a reader 11, a display device 12, a control terminal 13, and a processor 100, as the hardware configuration. However, the hardware configuration of the system 10 may not necessarily be limited to the preferred embodiment shown in FIG. 1. Various processes of the system 10 may be implemented by cooperative combinations of software and hardware in the control terminal 13 and the processor 100, for example.

As illustrated in FIG. 1, the system 10 proposed herein includes a first memory 101 to a fifth memory 105, and a first processor 201 to a sixth processor 206. Other than the first memory 101 to the fifth memory 105 and the first processor 201 to the sixth processor 206 as will be described herein, the system 10 may include any additional storage or memory, and/or any additional processor as required.

Of these memories, the first memory 101 is provided to store user IDs associated in a one-to-one correspondence with users.

The second memory 102 is provided to store instrument IDs associated in a one-to-one correspondence with surgical instruments.

The third memory 103 is provided to store work information and work hours of work performed on the instruments each identified by a corresponding one of the instrument IDs, in association with the instrument IDs and the user IDs.

The first processor 201 is configured or programmed to extract work information of one of the users identified by a corresponding one of the user IDs in a predetermined period Z1, based on the user IDs and the work hours stored in the third memory 103.

The above-described system 10 is able to extract work information of the users in a predetermined period Z1.

The system 10 suitably records the work history of the instrument identified by an instrument ID in each of the steps of the following cycle: surgical operation→collection (sorting)→cleaning→assembling (sealing)→sterilization→storage→surgical operation, for example. The system 10 is further configured or programmed so that the work history of each of the surgical instruments may include work information about repair work when appropriate. Hereinafter, an example of an application of the system 10 will be illustrated.

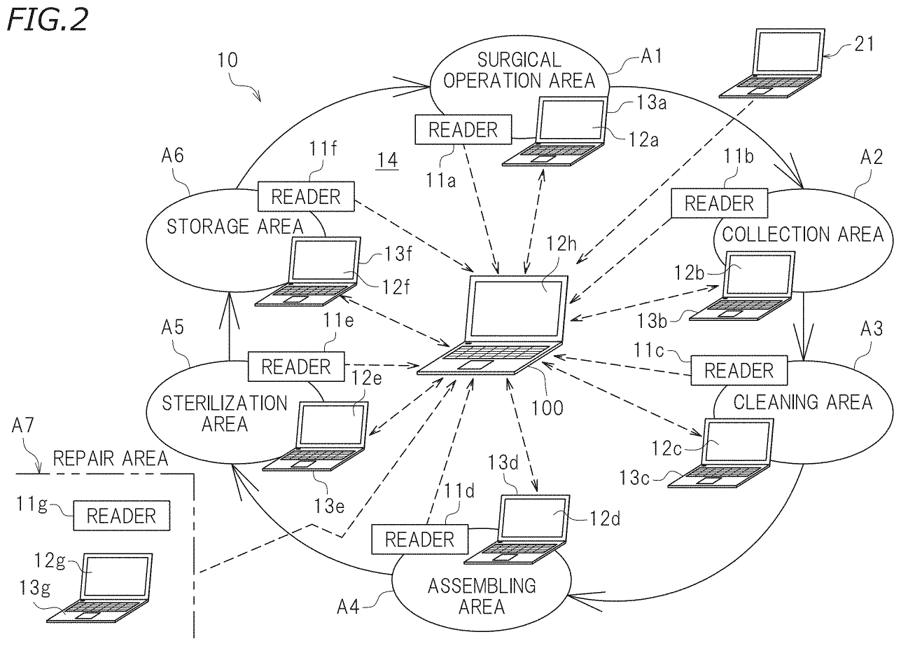

FIG. 2 is a schematic view illustrating an example of an application of the system 10. The preferred embodiment shown in FIG. 2 includes separate areas A1 to A7, namely, a surgical operation area A1, a collection area A2, a cleaning area A3, an assembling area A4, a sterilization area A5, a storage area A6, and a repair area A7.

In the system 10, identifiers, which may also be referred to as “area IDs”, are assigned in associated with the respective areas A1 to A7. The area IDs are stored in the master data in association with the respective areas A1 to A7.

The surgical operation area A1 is an area in which the instruments and the surgical instrument sets are used by doctors and nurses, and an area in which a surgical operation and a medical examination are carried out.

The collection area A2 is an area in which the surgical instrument sets and the surgical instruments are collected from the surgical operation area A1. Here, the surgical instruments may be collected separately, surgical instrument by surgical instrument.

The cleaning area A3 is an area in which the surgical instrument sets and the surgical instruments are cleaned. Here, the surgical instruments may be disassembled into components for cleaning.

The assembling area A4 is an area in which the cleaned surgical instrument sets and the cleaned surgical instruments are assembled and packaged into predetermined containers or bags.

The sterilization area A5 is an area in which the surgical instrument sets and the surgical instruments are sterilized. The surgical instrument sets and the surgical instruments are sterilized as they are kept packaged in predetermined containers or bags.

The storage area A6 is an area in which the sterilized surgical instrument sets and the sterilized surgical instruments are stored.

The repair area A7 is an area in which the instruments and the surgical instrument sets are repaired or inspected.

The work carried out in the repair area A7 is not limited to repairs, but may include maintenance work, such as inspections. Thus, when a surgical instrument is sent out for repair or inspection, the system 10 does not make a distinction between repair and inspection, but classifies the status of the surgical instrument into the concept of repair area.

Note that this preferred embodiment includes the surgical operation area A1, the collection area A2, the cleaning area A3, the assembling area A4, the sterilization area A5, the storage area A6, and the repair area A7, but this is merely illustrative.

Each of the above-described areas A1 to A7 can be defined as a location where a predetermined work is performed, but each of the areas A1 to A7 does not always match the location where a predetermined work is performed. For example, in the surgical instrument management system, it is possible to omit some of the areas, such as the surgical operation area, that does not involve any work performed by the users who are the subject of the work management.

For example, the work processes such as cleaning, assembling, sterilization, and repair (repair and inspection) may be carried out by a specialist contractor, unlike surgical operations. In such cases, the cleaning area A3, the assembling area A4, the sterilization area A5, and the repair area A7 may be provided in a different facility from the facility that contains the surgical operation area A1, the collection area A2, and the storage area A6. It is also possible that the surgical operation area A1, the collection area A2, and the storage area A6 may be provided in different locations.

The system 10 is able to collectively manage the surgical instrument sets and the instruments that are used in a plurality of hospitals. In such cases, the areas A1 to A7 may be provided in each of the plurality of hospitals. Thus, each of the areas A1 to A7 is not limited to a single location.

In particular, the repair work may in some cases be carried out at a location outside a hospital. For example, instruments and surgical instrument sets may suffer various damages resulting from use, such as damaged cutting edges and partial smoothness in their joints, engagements, and moving parts of the instruments such as forceps. In such cases, the repair is outsourced to outside contractors.

In the preferred embodiment shown in FIG. 2, the repair area A7 is set to be a single area, but the location where the repair area A7 is set is not limited to a single location. In reality, it is possible that the outsourced contractors may be different depending on the type of work, such as repair and inspection.

In this preferred embodiment, as illustrated in FIG. 2, the readers 11a to 11g, each defining and functioning as a reading device, and the control terminals 13a to 13g are arranged respectively in the areas A1 to A7. In the system 10, the readers 11a to 11g and the control terminals 13a to 13g, which are disposed in the respective areas A1 to A7, may be associated respectively with the areas A1 to A7. For example, the system 10 may be configured or programmed so that the information that is input with the readers 11a to 11g or with the control terminals 13a to 13g may be recorded as the information that is input at the respective areas A1 to A7 in association with the IDs assigned to the respective areas A1 to A7.

As illustrated in FIG. 2, the system 10 is implemented by the processor 100, the control terminals 13a to 13g, and the readers 11a to 11g. The control terminals 13a to 13g and the readers 11a to 11g are disposed respectively in the areas A1 to A7. The control terminals 13a to 13g and the processor 100 respectively include display devices 12a to 12h. In the present description, the reader(s), the display device(s), and the control terminal(s) may be simply and collectively referred to as the “reader 11”, the “display device 12”, and the “control terminal 13” when it is unnecessary to make a distinction between the readers 11a to 11g, between the display devices 12a to 12h, and between the control terminals 13a to 13g.

Each of the readers 11a to 11g disposed in the respective areas A1 to A7 is a device that reads an item-identifying information symbol incorporated in each instrument of surgical instrument sets, an item-identifying information symbol incorporated in each component part of each instrument, or an item-identifying information symbol affixed to a bag or a container that encloses instruments or surgical instrument sets.

Here, the item-identifying information may be a two-dimensional symbol in a predetermined form, for example. Examples of the item-identifying information used for the instruments in a surgical instrument set may include two-dimensional bar codes, contactless tags such as RFIDs, and engraved marks formed in the instrument surfaces. The engraved marks may be formed by laser engraving or impact engraving, for example.

Formation of the two-dimensional symbol by impact engraving may be performed by using an impact printer (e.g., MPX-95 manufactured by Roland DG Corp.), for example. Such an impact printer is capable of forming a data matrix with a very small size of, for example, about 1 mm square to about 4 mm square. The impact-engraved two-dimensional symbol is formed by indenting the surface of the instrument. The impact-engraved two-dimensional symbol is unlikely to damage, for example, a plated coating film on the surface of the instrument, so the impact-engraved two-dimensional symbol does not cause the instrument to rust easily. Moreover, due to the development of impact printer or the like, impact engraving of two-dimensional symbols can be applied to the existing instruments on which no two-dimensional symbol has been formed, and two-dimensional symbols can be set by the user and formed on the existing instruments.

In the assembling area A4, the sterilization area A5, or the storage area A6, each of the instruments is individually packaged appropriately, and each of the surgical instrument sets is collectively enclosed in a bag or a container, for example. In this case, each of the bags or the containers for enclosing an instrument or a surgical instrument set may be provided with item-identifying information, such as a two-dimensional symbol.

Each of the readers 11a to 11g may detect the two-dimensional symbol and may include cameras and CCD image sensors. The item-identifying information assigned to each instrument or each component part can be read by directing the item-identifying information symbol (which may be an impact-engraved two-dimensional symbol, for example) attached to each instrument or each component part toward the reading unit of one of the readers 11a to 11g. When the instrument or the surgical instrument set is enclosed in a bag or a container, the item-identifying information can be read by directing the item-identifying information attached on the bag or the container toward a desired one of the readers 11a to 11g.

Although each of the control terminals 13a to 13g and the processor 100 is depicted as a laptop terminal in FIG. 2, the type of terminal is not limited thereto. Each of the terminals may be a tablet terminal or a desktop terminal, for example.

The control terminals 13a to 13g may be configured or programmed to cooperate with the processor 100 to implement various processes of the system 10. In this preferred embodiment, the processor 100 may be configured or programmed to define and function as a host computer that defines and functions as a backbone of the processes of the system 10. Each of the control terminals 13a to 13g may cooperate with the processor 100 and function as a client computer of the system 10. The processor 100 and the control terminals 13a to 13g include memory storage devices, and the memory storage devices of the processor 100 and the control terminals 13a to 13g may be connected to each other via a communication network 14. The memory storage devices of the processor 100 and the control terminals 13a to 13g may be configured or programmed so that information can be written into and retrieved from the memory storage devices.

Each of the control terminals 13a to 13g and the processor 100 includes an arithmetic device, also referred to as a processor, CPU (central processing unit), or MPU (micro-processing unit), and a memory storage device (such as a memory and a hard disk). The functions of the control terminals 13a to 13g and the processor 100 are embodied by cooperative combinations with software stored in the memory storage device. For example, the configurations and the processes of the control terminals 13a to 13g and the processor 100 may be embodied as processing modules that perform predetermined computing tasks, data, or part thereof. Specifically, each of the processing modules includes a program, for example.

Referring to FIG. 1, each of the processor 100 and the control terminals 13a to 13g includes a memory storage area 120 for storage of data and a memory storage area 140 for storage of processing modules.

The memory storage area 120 for storage of data stores various kinds of master data and various data acquired by the readers 11a to 11g and the control terminals 13a to 13g, in addition to the above-described memories 101 to 105.

The memory storage area 140 for storage of processing modules stores processing modules of the system 10 as appropriate. The processing modules stored in the memory storage area 140 are not limited to the foregoing processors 201 to 210. The processor 100 and the control terminals 13a to 13g may be configured or programmed to, for example, call and execute the processing modules stored in the memory storage area 140. Herein, the memory storage area 120 that stores data may be constructed by, for example, a separate data server that is independent of the processor 100 and the control terminals 13a to 13g.

Each of the control terminals 13a to 13g and the processor 100 may be embodied by a single device, or may exhibit the functions as a processor by cooperative combinations of a plurality of devices. That is, although FIG. 1 depicts that the processor 100 includes one single device, the processor 100 is not limited to being one single device.

The control terminals 13a to 13g and the processor 100 respectively include display devices 12a to 12h, each of which functions as a display. The display devices 12a to 12h are configured or programmed to display images according to programs incorporated in the control terminals 13a to 13g and the processor 100. Each of the display devices 12a to 12h may include a touchscreen. When each of the display devices 12a to 12h includes a touchscreen, the system 10 may be configured or programmed to be operated by the user's touching the screen of the display device 12, for example.

The readers 11a to 11g, the control terminals 13a to 13g, and the processor 100 may be bidirectionally date-communicably connected to each other. In the preferred embodiment shown in FIG. 2, the readers 11a to 11g, the control terminals 13a to 13g, and the processor 100 cooperate with each other in such a state as to be capable of making data communication with each other through the communication network 14. The control terminals 13a to 13g, the readers 11a to 11g, and the processor 100 may be connected in such a state as to be able to make information communication with each other via, for example, a wireless communication network through a router (not shown) or a wired communication network through LAN cables.

Each of the displays 12a to 12g of the control terminals 13a to 13g as well as the display 12h of the processor 100, which are shown in FIG. 2, functions as the display device 12 shown in FIG. 1. In the preferred embodiment shown in FIG. 2, the control terminals 13a to 13g and the processor 100 are cooperated with each other to function as the processor 100 of the system 10 (see FIG. 1). In the preferred embodiments shown in FIGS. 1 and 2, the system 10 preferably is embodied by a plurality of devices. In the system 10, a plurality of personal computers, such as the control terminals 13a to 13g and the processor 100, cooperate with each other. The system 10 may be used simultaneously in parallel at a plurality of remote locations by a plurality of different users. A large number of surgical instrument sets and a large number of instruments may be managed in parallel in a plurality of steps.

FIG. 2 shows that one reader 11, one display device 12, and one control terminal 13 are arranged in each of the areas A1 to A7. However, the arrangement of the reader 11, the display device 12, and the control terminal 13 is not limited to the preferred embodiment shown in FIG. 2.

It is possible that the actual locations of the areas A1 to A7 may be overlapped with each other. For example, when collection is carried out at the same location as where a surgical operation or a medical practice is carried out, the location of the surgical operation area A1 and the location of the collection area A2 may be overlapped with each other. In this case, it is also possible that the reader 11, the display device 12, and the control terminal 13 may be used in common between the surgical operation area A1 and the collection area A2. In addition, it is possible that the functions and settings of the control terminal 13 may be switched from one to another so as to be suitable for the areas A1 to A7 by, for example, a process on software. For example, the control terminal 13 may be configured or programmed to be able to switch between the functions and settings suitable for the surgical operation area A1 and the functions and settings suitable for the collection area A2, as appropriate, by a predetermined operation.

The system 10 may further include further larger numbers of readers 11, display devices 12, and control terminals 13. On the contrary, the reader 11, the display device 12, and the control terminal 13 may not be arranged in all of the areas A1 to A7. It is also possible that different areas among the areas A1 to A7 may share a reader 11, a display device 12, and a control terminal 13.

For example, in a small-scale hospital, the cycle of surgical operation→collection (sorting)→cleaning→assembling (sealing)→sterilization→storage is performed in a limited space. In such cases, various types of functions performed by the readers 11a to 11g, the control terminals 13a to 13f, and the processor 100 may be aggregated into a single reader and a single control terminal. Thus, the system 10 may be configured or programmed as a stand-alone apparatus. On the other hand, in a large-scale hospital that has a large number of operating rooms, the system 10 may be configured so that the processor 100 is connected bidirectionally communicatively with the control terminals 13 in the respective areas. In this case, the system 10 may be built as a client-server system in which the processor 100 defines and functions as a host server and each of the control terminals 13 of the respective areas defines and functions as a client. Alternatively, the system 10 may be built as a cloud-based system operated in a plurality of hospitals and facilities that are remote from each other. In this case, the system 10 may be built as such a system that, for example, the processor 100 defines and functions as a data center in which various types of data are aggregated, and various types of functions of the system 10 are usable from control terminals 13 arranged in a plurality of remote hospitals and facilities through, for example, a web browser.

In the preferred embodiment shown in FIG. 2, the reader 11a and the control terminal 13a are located in the surgical operation area A1. However, the reader 11a and the control terminal 13a may not be provided in the surgical operation area A1. For example, as long as the storage area A6 and the collection area A2 are provided with the readers 11b and 11f and the control terminals 13b and 13f, the surgical operation area A1 need not be provided with the reader 11a or the control terminal 13a. In this case, the work information at the surgical operation area A1 may be input at the storage area A6 and the collection area A2.

Some processes such as cleaning work, assembling work, sterilization work, and repair work may be carried out by a specialist contractor outside the hospital. It is possible that such an outside contractor is not involved in the system 10. This means that some areas such as the cleaning area A3, the assembling area A4, the sterilization area A5, and the repair area A7 may not be provided with the reader 11, the display device 12, or the control terminal 13. Among them, the cleaning area A3, the assembling area A4, and the sterilization area A5 are within the normal use cycle, surgical operation→collection (sorting)→cleaning→assembling (sealing)→sterilization→storage. This means that these work processes are performed regularly, so the contents and the costs are foreseeable.

On the other hand, the repair work, such as repairing of instruments and surgical instrument sets, is out of the normal use cycle, surgical operation→collection (sorting)→cleaning→assembling (sealing)→sterilization→storage, and it can occur irregularly or unexpectedly. Therefore, the contents and the costs of the repair work that can arise in the future are difficult to foresee.

For the repair work, surgical instrument sets and instruments are sent from the collection area A2 and the assembling area A4, for example, for repair and inspection.

It is possible that the repair area A7 may be set in an outside specialist contractor and the repair area A7 may not be provided with the reader 11 or the control terminal 13. Accordingly, regardless of the preferred embodiment shown in FIG. 2, the repair area A7 may not be provided with the reader 11g or the control terminal 13g.

For example, instruments and surgical instrument sets are sent to the repair area A7 from another area. Then, when the work to be performed at the repair area A7 is completed, the instruments and the surgical instrument sets are returned from the repair area A7 to the other area. This means that, if the other area is provided with a reader 11 and a control terminal 13, it is possible to manage the information about sending of surgical instrument sets and instruments to the repair area A7 and the information about the work contents performed at the repair area A7 can be input at the area from which the instruments and the surgical instrument sets are sent out, or at the area to which the instruments and the surgical instrument sets are returned. For inputting the work information, this also applies to the case where the work processes to be performed at the cleaning area A3, the assembling area A4, and the sterilization area A5 are outsourced to outside contractors. Specifically, the work information may be input at the area to which the instruments and the surgical instrument sets are returned from the cleaning area A3, the assembling area A4, and the sterilization area A5.

As illustrated in FIG. 1, the system 10 includes, for example, the first memory 101 to the fifth memory 105 and the first processor 201 to the sixth processor 206.

In the system 10, the processes to be performed by the processors may be processed by any of the processor 100 and the control terminals 13a to 13g. The information stored in the memories may be managed by the processor 100 as a host computer, for example, and may be shared by the control terminals 13a to 13g.

The first memory 101 stores user IDs associated in a one-to-one correspondence with users. That is, in this system 10, the term “user” is intended to mean a “worker”, for example. Each of the user IDs is assigned to a respective one of the workers in order to identify the workers in a one-to-one relationship. The user IDs may be configured so that they can be identified by a two-dimensional code attached a name tag distributed to each of the workers, or by using biometric authentication of each individual worker.

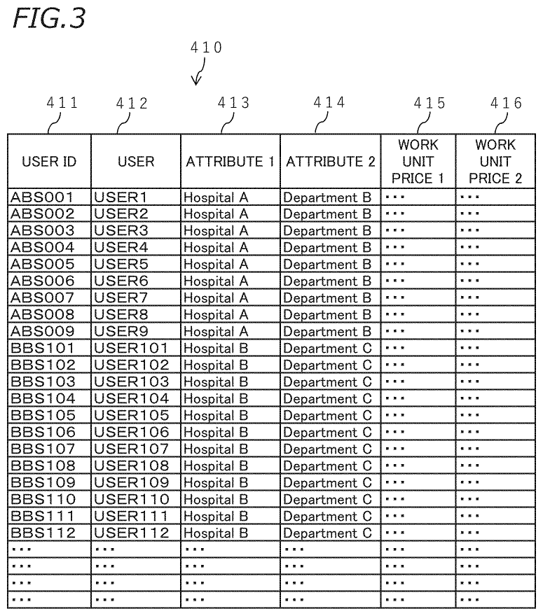

FIG. 3 is a view illustrating an example of the configuration of a database defining and functioning as a first memory 101 that stores user IDs. The database 410 shown in FIG. 3 may include a column 411 to store user IDs, a column 412 to store user names, columns 413 and 414 to record attributes, and columns 415 and 416 to record work unit prices. A plurality of columns may be provided to record attributes. Each of the columns 413 and 414 may record, for example, a hospital or a medical department to which a user belongs. The columns 415 and 416 to record work unit prices may record the work unit prices of the users. Each of the work unit prices may be a unit price that is set per unit time, or may be a unit price that is set for a predetermined work. When the unit price is assessed by the proficiency level or the job position of the worker, it is possible to record the indicators to assess the proficiency level of the workers, the job positions of the workers, and the like. An example of the unit price for a predetermined work may be a unit price for the work of using one of the readers 11a to 11g to read the item-identifying information of each of the surgical instruments.

The second memory 102 stores instrument IDs associated in a one-to-one correspondence with surgical instruments.

In this system 10, each of the instrument IDs corresponds in a one-to-one correspondence to a specific instrument. In this preferred embodiment, as mentioned above, each of the instruments is provided with item-identifying information that is readable by the readers 11a to 11g. The second memory 102 may store the item-identifying information assigned to each of the instruments and the instrument ID of each of the instruments in association with each other. The system 10 may be configured or programmed to be able to identify the instrument ID associated in a one-to-one correspondence with the instrument when the item-identifying information is read by one of the readers 11a to 11g.

In some cases, each of the instruments may further include a plurality of component parts. In the system 10, the component parts that constitute each of the instruments are also provided with parts IDs. Each of the parts IDs corresponds in a one-to-one correspondence to a specific instrument and a component part of the instrument. The system 10 is also able to identify the instruments of each of the surgical instrument sets and the component parts of each instrument with parts IDs. In this case, each of the component parts may be also provided with item-identifying information. The system 10 may also be configured or programmed so that, when the item-identifying information is read by one of the readers 11a to 11g, the system 10 can identify the parts ID and the instrument ID that are associated in a one-to-one correspondence with each of the component parts. Thus, each of the instruments and the parts is identified by causing the reader 11 to read the item-identifying information assigned to each of the instruments and the parts. Thus, the work information can be input correctly for each individual items of the instruments or component parts even when there are a plurality of instruments of the same type and a plurality of component parts of the same type.

The third memory 103 is configured to store work information and work hours of work performed on the instruments each identified by a corresponding one of the instrument IDs, in association with the instrument IDs and the user IDs.

Herein, the term “work information” refers to information about various types of work, and the work information may be encoded according to the work contents. The work contents and the encoded information may be stored in the master data. In such a third memory 103, the work content and the work date and time may be stored in association with a user ID, for example.

The fourth memory 104 stores set IDs associated in a one-to-one correspondence with surgical instrument sets each including a plurality of instruments. That is, in this system 10, the surgical instrument sets are provided with set IDs that are associated in a one-to-one correspondence with the surgical instrument sets so that each of the surgical instrument sets can be identified by a set ID.

In this preferred embodiment, the set IDs are stored in such a manner that the set IDs associated in a one-to-one correspondence with the surgical instrument sets are further associated with the instrument IDs of the instruments that are included in the corresponding surgical instrument sets and also with the parts ID of the component parts that are included in the corresponding surgical instrument sets. That is, when a set ID is identified, it is possible to identify the instrument IDs of the instruments included in the surgical instrument set identified by the set ID as well as the parts IDs of the component parts included in the surgical instrument set identified by the set ID. On the other hand, when an instrument ID is identified, it is possible to identify the set ID of the surgical instrument set that includes the corresponding instrument.

In the case where an instrument includes component parts, the parts IDs of the component parts included in the instrument are identified from the instrument ID. Also, when a parts ID of a component part is identified, the instrument ID of the instrument that includes the component part is identified accordingly. Moreover, in the case where when the instrument is included in a surgical instrument set, it is possible to identify the set ID of the surgical instrument set that is associated with the parts ID when the parts ID of a component part is identified. For example, the second memory 102 may store the item-identifying information assigned to each instrument, instrument IDs, and set IDs in association with each other. The system 10 may be configured or programmed to be able to identify the instrument ID and the set ID when item-identifying information is read by one of the readers 11a to 11g. Furthermore, the system 10 may be configured or programmed to be able to identify the corresponding set ID when the item-identifying information assigned to a component part is read by one of the readers 11a to 11g.

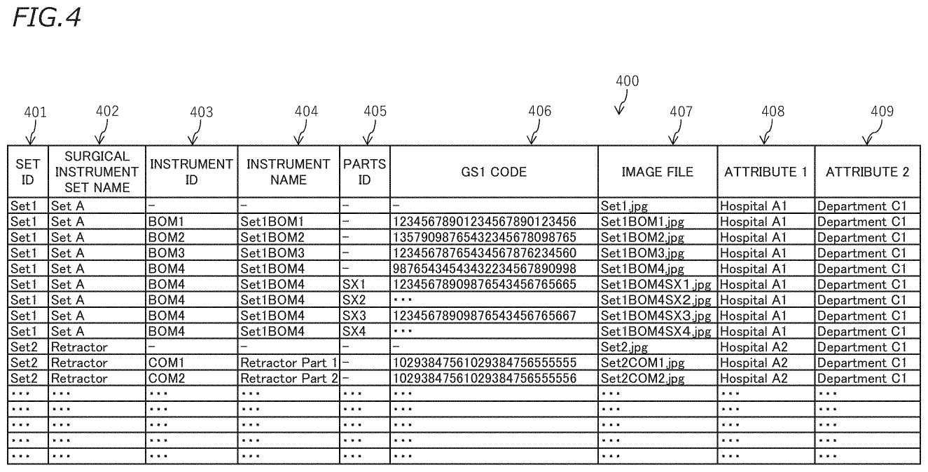

Herein, the second memory 102, which stores instrument IDs, and the fourth memory 104, which stores set IDs, may include respective independent databases. Alternatively, the second memory 102 and the fourth memory 104 may include a single database. FIG. 4 is a view illustrating an example of the configuration of a database 400 in which a second memory 102 and a fourth memory 104 are integrated into a single database. FIG. 4 shows that there are provided a column 401 that stores set IDs, a column 402 that stores surgical instrument set names; a column 403 that stores instrument IDs; a column 404 that stores instrument names; a column 405 that stores parts IDs; a column 406 that stores GS1 codes as item-identifying information; a column 407 that stores image files of surgical instrument sets, instruments, and component parts; and columns 408 and 409 that store information related to owners of instruments and surgical instrument sets. These IDs and pieces of information are stored in association with each other. Each of the image files contains an image of a surgical instrument set, an instrument, or a component part. The system 10 is able to be configured or programmed to display the image files on the display device 12 (see FIG. 1).

As in the preferred embodiment shown in FIG. 4, for the instruments included in the surgical instrument sets, the second memory 102 may additionally store set IDs of the surgical instrument sets respectively associated with the instrument IDs. As a result, when the instrument ID of an instrument included in a surgical instrument set is identified, the set ID of the surgical instrument set that includes the instrument is accordingly identified.

In addition, attribute information is stored in association with the instrument IDs and the set IDs. Herein, in cases of hospitals, for example, the attribute information may be specified as information for identifying a medical department, such as surgery and internal medicine. Moreover, when the instruments and the surgical instrument sets of a plurality of hospitals need to be managed collectively, the attribute information includes information for distinguishing one hospital from another. Thus, the attribute information may include a plurality of types of information at some hierarchical levels. For example, in the example shown in FIG. 4, attribute 1 includes information indicating a hospital, and attribute 2 includes information indicating a medical department. The attribute information is not limited thereto, and may include different types of information other than hospitals and medical departments, such as the manufacturer and the model of the instrument or the surgical instrument set. Thus, the attribute information may be information for classifying instruments and surgical instrument sets.

The fifth memory 105 stores work information and outsourcing unit prices in association with each other.

As mentioned earlier, some processes such as cleaning work, assembling work, sterilization work, and repair work may be carried out by a specialist contractor outside the hospital. The fifth memory 105 may store the work information about the work to be outsourced to an outside contractor and the outsourcing unit price of the work so that the work information and the outsourcing unit price are associated with each other. Here, the outsourcing unit price may be a cost that is set for the work to be outsourced to the outside contractor.

As illustrated in FIG. 4, the system 10 may be provided with pre-stored master data, which include set IDs, instrument IDs, and parts IDs, as well as associated information therewith, such as item-identifying information, image data, and attribute information. The information that is stored in the master data is not limited to the items listed in FIG. 4. For example, the user IDs for identifying the workers stored in the first memory 101 may also be stored in advance in the master data of the system 10. The user IDs may also be configured so that they can be identified by a type of information that is readable with the reader 11b.

Each of the areas A1 to A7, the readers 11a to 11g, the control terminals 13a to 13g, and the processor 100 may also be provided with item-identifying information assigned thereto, which may also be stored in advance in the master data of the system 10.

Moreover, the pre-stored master data of the system 10 may also include information indicating cleaning methods, packaging materials used in sterilization and storage, sterilization methods, and the like, as appropriate.

The pre-stored master data may also include the work information and the outsourcing unit prices, which are to be stored in the fifth memory 105.

In this preferred embodiment, the readers 11a to 11g are arranged respectively in the areas A1 to A7, each being a predetermined work space. Each of the instruments possesses item-identifying information. In the first memory 101, the item-identifying information, the instrument ID, and the set ID are stored in association with each other for each of the instruments. The work information of each instrument or each surgical instrument set may be input after the instrument or the surgical instrument set has been identified by reading the item-identifying information with one of the readers 11a to 11g.

In this preferred embodiment, the fifth processor 205 may be configured or programmed to, when item-identifying information is detected by one of the readers 11a to 11g, identify one of the instrument IDs by the item-identifying information, and store the one of the instrument IDs into the third memory 103 in association with work information and work hours of work performed on one of the instruments that is identified by the one of the instrument IDs and with the user IDs. The fifth processor 205 may also be configured or programmed to be able to identify one of the set IDs when item-identifying information is detected by one of the readers 11a to 11g. In this case, the fifth processor 205 is also configured or programmed to add the information about the work performed on the surgical instrument set to the work history of the surgical instrument set in association with the identified set ID. This allows the system 10 to identify an instrument ID and a set ID from the item-identifying information when the item-identifying information is detected by one of the readers 11a to 11g. As a result, even when it is necessary to manage a plurality of instruments of the same type and a plurality of surgical instrument sets of the same type, it is possible to identify an individual one of the instruments or the surgical instrument sets and input the work information of the individual one of the instruments or the surgical instrument sets.

As illustrated in FIG. 2, the system 10 includes the readers 11a to 11g arranged in the respective areas A1 to A7. The system 10 may be configured or programmed so that, by using the readers 11a to 11g, the user IDs can be identified through the two-dimensional codes attached name tags distributed to the workers, or through biometric authentication of each individual worker.

Also, the item-identifying information of the surgical instruments is detected by the readers 11a to 11g, and the instrument IDs are identified by the detected item-identifying information.

In the areas A1 to A7, user IDs and instrument IDs are read by the readers 11a to 11g. The instrument IDs, the user IDs, and the work information and the work hours may be stored into the third memory 103 so that the instrument IDs, the user IDs, and the work information and the work hours are associated with each other. The work information indicates the work performed on the instruments identified by the instrument IDs. The work hours indicate the length of time of the work performed on the instruments identified by the instrument IDs.

Because the surgical instruments and the time of work are identified in this way with the use of the readers 11a to 11g, the work information and the work hours of the work performed on the surgical instruments can be entered in a more reliable manner. Note that, in the areas A1 to A7, the worker may manually enter the work information or part of the work information by using the respective control terminals 13a to 13g.

Although the items of the work information to be recorded may vary between the areas A1 to A7, the work information may be recorded in, for example, a predetermined format. In addition, the items of the work information recorded in the areas A1 to A7 may be configured so that they can be integrated with each other for predetermined items.

In the surgical operation area A1, for example, when a stored instrument or a stored surgical instrument set is brought into the surgical operation area A1, item-identifying information is detected by the reader 11a and the instrument ID or the set ID is identified. Then, the information indicating the date on which the instrument or the surgical instrument set is delivered and the information during use are recorded in association with the instrument ID or the set ID. The work information recorded at the surgical operation area A1 may include, for example, the users (workers) such as doctors, patients, medical record numbers of the patients, surgical operations, and consultation numbers. This enables the system 10 to store the work history indicating what kind of surgical operation the instrument or the surgical instrument set was used for. As a result, the amount of information that can be tracked by the work history of the instrument or the surgical instrument set increases. It is also possible that the system 10 may import information contained in an existing electronic patient record into the work information obtained at the surgical operation area A1. It is also possible that the instrument IDs or the set IDs of the instruments or the surgical instrument sets that have been used in a surgical operation may be written into the existing electronic patient record. Thus, it is possible to construct a system that the existing electronic patient record and the system 10 cooperate with each other.

The instruments and the surgical instrument sets used that were used in the surgical operation area A1 are brought into the collection area A2. In the collection area A2, the item-identifying information is detected by, for example, the reader 11b, and the instrument IDs and the set IDs are identified. The work information recorded at the collection area A2 may include information such as the dates and times of collection and the user IDs of the workers who performed the collection, for example.

In the collection area A2, the display device 12b may display a list of the instruments included in a surgical instrument set, for example. While the reader 11b is detecting the item-identifying information of the instruments included in the surgical instrument set, the instruments may be collected. When the item-identifying information of the instruments included in the surgical instrument set is detected, the instruments can be identified on the display device 12b. It is also possible that, when the worker specifies an instrument displayed on the display device 12b in the collection work, the display device 12b may dim the indication of the instrument for which the item-identifying information was detected from the list of the instruments included in the surgical instrument set. In this case, the display device 12b may include a touchscreen. The display device 12b may be configured so that, when the worker touches an indication representing the instrument to be collected (in other words, the instrument identified by the item-identifying information) from the list of the instruments displayed on the display device 12b, the indication of the instrument is changed. By such a process, the worker may be allowed to easily recognize visually that the subject instrument has been collected properly. Then, when all the instruments included in the surgical instrument set used in the surgical operation area A1 have been collected in the collection area A2, the collection of the surgical instrument set is completed. At that time, it is possible that the worker may be allowed to easily recognize visually that the subject instruments have been collected properly by, for example, dimming the indication of the subject surgical instrument set or displaying an icon indicating the completion of collection on the display device 12b.

In the cleaning area A3, the instrument ID is identified when the item-identifying information of an instrument included in a surgical instrument set is detected by, for example, the reader 11c. The work information obtained at the cleaning area A3 may be stored in association with the identified instrument ID. The work information obtained at the cleaning area A3 may include, for example, information on the cleaning method, such as the cleaning machine and the detergent used for the cleaning. In the cleaning area A3, the user IDs that identify the workers of the cleaning work may be stored associated with the work information.

It is also possible that the instrument ID and a cleaning method may be associated with each other and stored in advance in the system 10. In this case, it is possible that when the reader 11c detects the item-identifying information of an instrument included in a surgical instrument set, the display device 12c in the cleaning area A3 may display a cleaning process required for the instrument on the screen based on the identified instrument ID. Because the instrument ID is identified by the reader 11c, it is unlikely that the cleaning process is performed for a wrong instrument or that the work information is input for a wrong instrument. The reader 11c may be provided for, for example, a cleaning machine. In that case, it is also possible that, while the reader 11c is reading the item-identifying information of the instrument, the instrument may be put into the cleaning machine. It is also possible that the information on the cleaning conditions and the like that are set for the cleaning machine may be recorded in the work information. Herein, examples of the cleaning method include manual cleaning, automated single-bath cleaning, automated multi-bath cleaning, ultrasonic cleaning, and chemical cleaning. Each of the cleaning methods may use a predetermined cleaning machine and a predetermined detergent in a predetermined way.

In addition, information contained in an existing electronic patient record may be imported into the work information obtained at the surgical operation area A1. In that case, the cleaning method and the sterilization method for the instrument may be recorded in the master data in advance, in association with information about patients and surgical operations. Accordingly, the system 10 may be constructed to appropriately identify a cleaning method required for the instrument based on the master data and the information about patients and surgical operations that is contained in the work information obtained at the surgical operation area A1. In this way, the system 10 may be constructed so as to identify a special cleaning method when such a special cleaning method is required by the instrument or the surgical instrument set based on the information provided by the electronic patient record. Likewise, in the sterilization area A5, the system 10 may be constructed so as to identify a special sterilization method when such a special sterilization method is required by the instrument or the surgical instrument set based on the information provided by the electronic patient record.

In the assembling area A4, various activities are performed, such as assembling of an instrument from cleaned parts, and collecting of instruments to be included in a surgical instrument set to form the surgical instrument set. In assembling the instrument, required lubricating oil may be applied to the instrument.

In the assembling area A4, when the item-identifying information of an instrument included in a surgical instrument set is detected by the reader 11d, for example, the instrument ID of the instrument and the set ID of the surgical instrument set are identified. Then, the list of the instruments included in the surgical instrument set is displayed. At this time, it is possible that the necessary assembling work that needs to be performed in the assembling area A4 may be displayed on the screen. For example, it is possible to display the lubricating oil to be applied at the time of assembling of the instrument when such is specified in advance.

Each of the instruments and the surgical instrument sets is enclosed in a predetermined bag or container. At that time, the packaging material and the packaging method may be recorded additionally. Examples of the packaging material include nonwoven fabric, sterilization bags, containers, and trays. Examples of the packaging method include wrapping the instrument or the surgical instrument set with nonwoven fabric, enclosing the instrument or the surgical instrument set in a sterilization bag, and enclosing the instrument or the surgical instrument set that is wrapped with nonwoven fabric in a sterilization bag. The surgical instrument set should be packaged while checking that all the instruments to be included in the surgical instrument set are present. In this case, it is possible that the instruments may be packaged while displaying the list of instruments to be included in the surgical instrument set on the display device 12d displays and detecting the item-identifying information of the instruments with the reader 11d when packaging the instruments. Also, it is possible to change the indication of the instrument that has already been packaged in the list of the instruments. When the indication of the packaged instrument changes on the display device 12d, the worker is able to easily confirm the instruments that have not yet been packaged. This prevents an instrument from missing from the surgical instrument set, and prevents a wrong instrument from being placed into the surgical instrument set.

The instruments and the surgical instrument sets are packaged, for example, in a bag or a container and hermetically sealed therein. The instruments that are not included in surgical instrument sets are packaged piece by piece independently. The surgical instrument set is packaged, for example, in a single collection of instruments. Each of the packaged bags or the packaged containers may be provided with item-identifying information that identifies the bag or the container. Then, the item-identifying information attached to the bag or the container may be stored in association with the instrument ID for identifying the instrument enclosed therein and the set ID for identifying the surgical instrument set enclosed therein. For example, in the packaging work, an instrument or a surgical instrument set is enclosed in a bag or a container while reading the individual item information attached on the bag or the container and the individual item information of the instrument or the surgical instrument set to be enclosed in the bag or the container. At this time, the system 10 may store the item-identifying information of the bag or the container in association with the item-identifying information of the instrument or the surgical instrument set that is enclosed therein. This makes it possible to identify the set ID of the surgical instrument set or the instrument ID of the instrument that is enclosed in the bag or the container in a post-process, based on the item-identifying information attached on the bag or the container.

Thus, in the assembling area A4, the work information obtained at the assembling area A4 is stored in association with the item-identifying information of the bag or the container, for example, which is read by the reader 11c. The work information obtained at the assembling area A4 may include the instrument IDs of the instruments enclosed in a bag or a container, the set IDs of the surgical instrument sets enclosed in a bag or a container, the workers, and the dates and times at which item-identifying information is read by the reader 11c in assembling work. Moreover, it is possible that the item-identifying information of the bag or the container and the item-identifying information of the instrument or the surgical instrument set enclosed therein may be stored in association with each other. Furthermore, in the assembling area A4, the user IDs that identify the workers of the assembling work may be stored associated with the work information.

In the sterilization area A5, the instruments and the surgical instrument sets are kept enclosed in a packaging material, such as a bag or a container, while they are handled. In the sterilization area A5, the item-identifying information attached to the bag or the container is read by, for example, the reader 11e. Then, the instrument or the surgical instrument set is identified based on the item-identifying information. Then, a predetermined sterilization treatment is performed for the instrument or the surgical instrument.

In this preferred embodiment, a database is prepared in advance in which a sterilization treatment is pre-stored in association with the instrument ID or the set ID. In the sterilization area A5, the item-identifying information attached to the bag or the container is read by the reader 11e, and the instrument ID or the set ID of the instrument or the surgical instrument set that is enclosed in the bag or the container is identified. The system 10 may be configured or programmed so that, based on the instrument ID or the set ID, the sterilization treatment to be performed at the sterilization area A5 is specified and displayed on the display device 12e.

This serves to allow the worker to easily understand the sterilization treatment that is to be performed for the instrument or the surgical instrument set enclosed in a bag or a container. In the sterilization area A5, the reader 11e may further be installed on a sterilizer, such as an autoclave. In that case, the instrument or the surgical instrument set may be placed into the sterilizer after the item-identifying information attached to the bag or the container is read by the reader 11e. This serves to allow the sterilization treatment to be performed under predetermined conditions (such as temperature and treatment time). In addition, the system 10 may be configured or programmed to prevent the worker from making errors by, for example, sounding an alarm in cases where the sterilizer in which the instrument or the surgical instrument set is to be placed is inappropriate or the conditions of the sterilization treatment that are set in the sterilizer are inappropriate when the item-identifying information attached to the bag or the container is read by the reader 11e.

In the sterilization area A5, the item-identifying information attached to the bag or the container is read by the reader 11e, for example. Then, the instrument ID or the set ID is identified in association with the item-identifying information attached to the bag or the container. Then, the work information obtained at the sterilization area A5 is stored in association with the instrument ID, the set ID, and the item-identifying information attached to the bag or the container. The work information obtained at the sterilization area A5 may include, for example, the sterilization method and conditions of the sterilization treatment. Specifically, the work information obtained at the sterilization area A5 may further include, for example, information indicating the sterilizer used for the sterilization treatment, conditions of the sterilization such as temperature and treatment time, the end date and time of the sterilization treatment, and the like. In the sterilization area A5, the user IDs that identify the workers of the sterilization work may be stored associated with the work information.

The storage area A6 stores the instruments and the surgical instrument sets after sterilization. In the storage area A6, the item-identifying information attached to each of the bags and the containers is read by the reader 11f. Then, the instrument ID or the set ID is identified in association with the item-identifying information attached to each of the bags and the containers. After the instrument IDs of the enclosed instruments and the set IDs of the enclosed surgical instrument sets are identified, the bags and the containers are sorted and stored in an appropriate location. The work information recorded at the storage area A6 may include, for example, information such as the storage location of each of the instruments and the surgical instrument sets and the date and time indicating when each of the instruments and the surgical instrument sets has been stored. Because the storage location of each of the instruments and the surgical instrument sets is recorded, it is easy to pick out the instrument or the surgical instrument set. Also, because the date and time when each of the instruments and the surgical instrument sets has been placed into storage is recorded, it is possible to calculate the period for which each of the instruments and the surgical instrument sets has been stored in the storage area A6. In addition, the worker IDs that identify the workers who have placed the instruments and the surgical instrument sets into the storage area A6 may be stored in association with the dates and times of the work of storing the instruments and the surgical instrument sets. In addition, the worker IDs that identify the workers who have removed the instruments and the surgical instrument sets from the storage area A6 may be stored in association with the dates and times of the work of removing the instruments and the surgical instrument sets.

In the repair area A7, repairs and inspections are carried out for the instruments and the surgical instrument sets. In the repair area A7, the reader 11g identifies the item-identifying information of an instrument or a surgical instrument set to identify the instrument ID or the set ID. The work information recorded at the repair area A7 includes, for example, processes such as repairs and inspections that have been performed for the instruments and the surgical instrument sets, the date and time when the instruments and the surgical instrument sets have been brought in, and the date and time when the instruments and the surgical instrument sets have been sent out. It is also possible to record the content, date, and time of the work performed in the repair area A7. The work information may also include the information about a factor that necessitates repair work.

Here, examples of the records of the factor necessitating repair work include damaged cutting edges of scalpels, improper engagement and non-smooth movement of forceps, and rust on the instruments. Furthermore, the information that may be recorded as the causes of improper engagement of forceps and the like may include long-term use, excessive force, dropping, and plating peeling. These factors that have necessitated the repair work may be coded into appropriate data in the master data that have been prepared in advance. Furthermore, the work information about the repair work may include information about the cost required for the repair work. The information recorded as the cost required for the repair work may be, for example, the amount of cost billed by the contractor to which the repair work was outsourced.

In this case, when work contents and billing data are digitized by the contractor to which the repair work is outsourced, the system 10 may be configured or programmed to read the work contents and billing data as the work information about repair work. In addition, when unit prices of the cost required for the repair work are predetermined for each type of instrument, each type of surgical instrument set, or each type of work content, the predetermined unit prices may be stored in the master data. In this case, the system 10 may be configured or programmed such that, when an instrument ID, a set ID, or a wok content is identified, the cost required for the repair work is obtained accordingly.

It should be noted that, as already mentioned previously, the repair area A7 may not be provided with the reader 11g or the control terminal 13g. When this is the case, one of the readers 11a to 11f or one of the control terminals 13a to 13f, which are provided in other areas, may be configured or programmed to identify the instrument or the surgical instrument set and to record processes such as repairs and inspections that have been performed for the instruments and the surgical instrument sets, the date and time when the instruments and the surgical instrument sets have been brought in, and the date and time when the instruments and the surgical instrument sets have been sent out. These items of information may be input by the reader 11 or the control terminal 13 that is installed in a supplying area from which the instruments and the surgical instrument sets are sent to the repair area A7, or in a destination area to which the instruments and the surgical instrument sets are sent from the repair area A7.

As has been described above, the work information to be recorded and the items to be recorded vary among the areas A1 to A7. The memory storage device of the system 10 may include, for example, tables (information storage tables) each in a predetermined format for storing the items of work information obtained at each of the areas A1 to A7. The items of the work information obtained at the areas A1 to A7 may be stored in the fields of the tables prepared respectively for the areas A1 to A7.

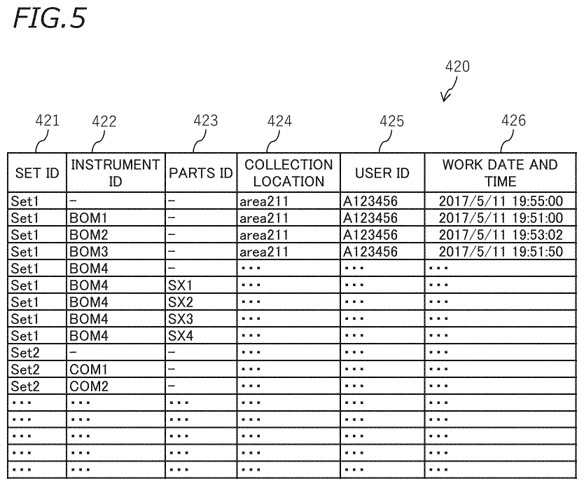

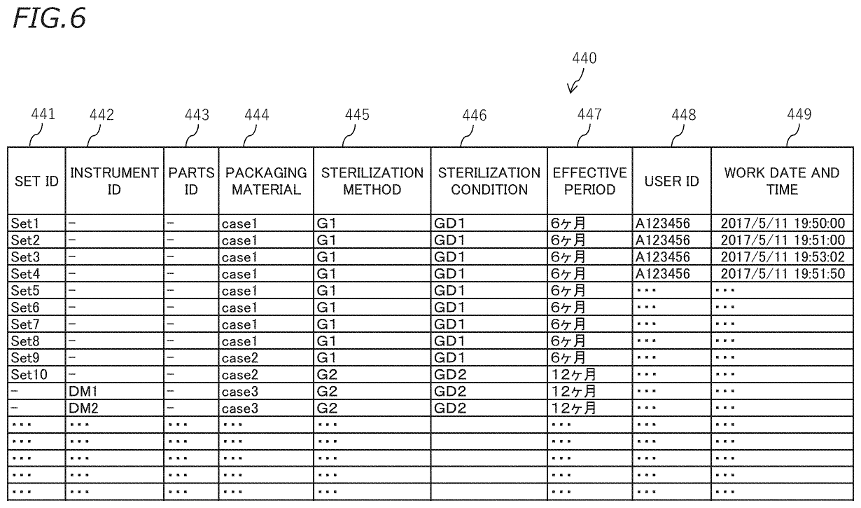

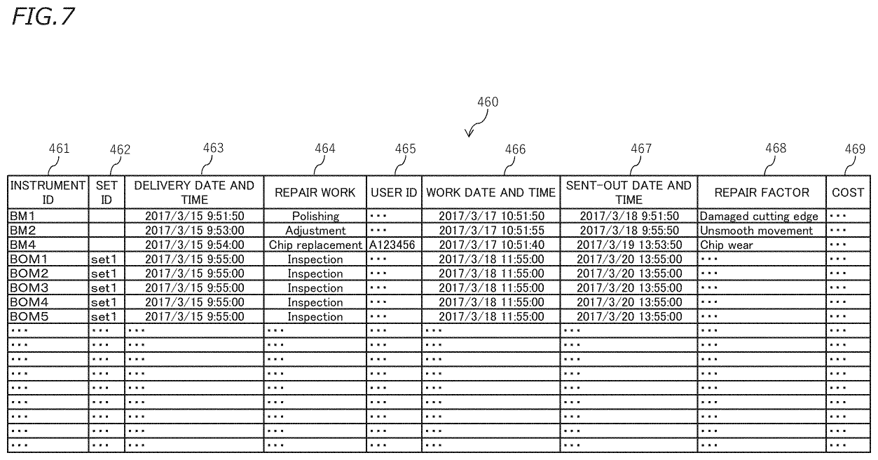

FIG. 5 is a view illustrating a table 420 that records work information for a collection area A2. FIG. 6 is a view illustrating a table 440 that records work information for a sterilization area A5. FIG. 7 is a view illustrating a table 460 that records work information for a repair area A7. These tables show examples of work information recorded at the respective areas.

In the collection area A2, the instruments of surgical instrument sets are collected from the surgical operation area A1 individually, for example. In the collection area A2, each of the instruments may be collected while the location of collection and the worker are identified and the item-identifying information symbol attached to the instrument is read with the reader 11b. Thus, as illustrated by the table 420 shown in FIG. 5, information is input into columns 421 to 426, which store set ID, instrument ID, parts ID, collection location, worker, and work date and time, respectively. Note that the records of the work information to be recorded at the collection area A2 are not limited to the example shown in FIG. 5. The records of the work information to be recorded at the collection area A2 may include further detailed information.

In the sterilization area A5, the item-identifying information assigned to a bag or a container is read by the reader 11e, for example, to identify the instrument ID of the instrument or the set ID of the surgical instrument set that is enclosed in the bag or the container. Then, in the sterilization area A5, the sterilization method and the sterilization conditions required for the surgical instrument set or the instrument identified by the set ID or the instrument ID are set for the sterilizer. Then, the surgical instrument set or the instrument should be placed into the sterilizer while causing the reader 11e to read the item-identifying information assigned to the bag or the container. For example, in the sterilization treatment, it is often the case that an indicator showing whether appropriate sterilization was performed may be fitted onto the packaging material that encloses the instrument or the surgical instrument set. In that case, the indicator may change its color, for example, under a predetermined condition. In the sterilization area A5, the indicator may be fitted onto the instrument or the surgical instrument set. The fitting of the indicator may also be carried out in the assembling area A4. The item-identifying information assigned to the bag or the container may be read by the reader 11e also when the bag or the container is removed from the sterilizer, and the end time of the sterilization treatment should be recorded.

Thus, as illustrated by the table 440 shown in FIG. 6, information is input to columns 441 to 449, which store set ID, instrument ID, parts ID, packaging material, sterilization method, sterilization condition, effective period, worker, and work date and time, respectively. The records of the work information to be recorded in the sterilization area A5 are not limited to the example shown in FIG. 6. The record of the work information to be recorded in the sterilization area A5 may include further detailed information. For example, the records of the work information obtained at the sterilization area A5 may include image data of the indicator after the sterilization treatment.

Here, a period of time for which the sterilization treatment remains effective is input into the column 447 that stores effective period.

The effective period of sterilization treatment may be determined from the sterilization method and the sterilization conditions. In this case, the relationship between sterilization method, sterilization conditions, and effective period may be recorded in advance in the master data of the system 10. It is also possible that the effective period of sterilization treatment may be determined from the sterilization method and the sterilization conditions. In that case, the relationship between packaging method, sterilization method, sterilization conditions, and effective period may be prerecorded in the master data of the system 10. For example, when the packaging method, the sterilization method, and the sterilization conditions are input at the sterilization area A5, the effective period may be accordingly input based on the master data.

The column 449 for work date and time may record, for example, the time at which the item-identifying information assigned to the bag or the container has been read by the reader 11e when the sterilization treatment was completed and the bag or the container was taken out of the sterilizer.

As illustrated in FIG. 7, for example, at the repair area A7, items of information are input into columns 461 to 469, which respectively record the following items of information about the instruments and the surgical instrument sets that have been brought into the repair area A7: the instrument IDs and the set IDs of the instruments and the surgical instrument sets, dates and times indicating when they have been brought into the repair area A7, dates and times indicating when they have been brought out from the repair area A7, repair factors that are the factors that have necessitated the repair work, and costs required for the repair work. It is also possible that any appropriate information other than the above-listed items of information may be input.

The processor 100 of the system 10 may be configured or programmed to collect the item-identifying information read by the readers 11a to 11g of the respective areas A1 to A7 and the work information stored associated with the item-identifying information. This enables the system 10 to collect the information of the instruments and the surgical instrument sets to be managed by the system 10 at the areas A1 to A7 and to collectively manage the information.

Thus, the system 10 may include the third memory 103 that stores the user IDs, the instrument IDs, work information and work hours of work performed on the instruments each identified by a respective one of the instrument IDs so that the instrument IDs, the work information, the work hours, and the user IDs are associated with each other.

Here, the third memory 103 may store the user IDs, the instrument IDs, work information and work hours of work performed on the instruments each identified by a respective one of the instrument IDs so that the instrument IDs, the work information, the work hours, and the user IDs are associated with each other. The third memory 103 need not be including a single database. The third memory 103 may include a database group including a plurality of databases, such as the databases shown in FIGS. 5 to 7, which store data including user IDs, dates and times of work, and work information recorded at the areas A1 to A7.

The system 10 may include, for example, processors as described below.

The first processor 201 extracts work information of one of the users identified by a corresponding one of the user IDs in a predetermined period Z1, based on the user IDs and the work hours stored in the third memory 103.

For example, when the predetermined period Z1 is set to be the most recent one week, the system 10 may be configured or programmed to extract the work information of the users identified by user IDs in the most recent one week based on the user IDs and the work hours stored in the third memory 103. Here, the predetermined period Z1 is not limited to the most recent one week and may be set to any desired period, such as the most recent one month, the most recent one year, the last week, the week before last, the last month, the month before last, and so forth. In addition, the start date of calculation, the end date of calculation, and the like are not limited to recent dates either, and may also be set to any desired date.

This allows the system 10 to extract and provide the work contents of users in a predetermined period. In this case, the first processor 201 may be configured or programmed to obtain the total work hours of users in a predetermined period Z1. The first processor 201 may also be configured or programmed to obtain the proportions of the work hours for each of the users in a predetermined period Z1.

In this preferred embodiment, the system 10 includes the third processor 203 configured or programmed to set the period Z1. The third processor 203 may be configured or programmed to allow the user of the system 10 to input the period Z1 with a display device. The period Z1 may be selected from predetermined periods, such as 1 month, 3 months, 6 months, and 1 year, for example. Alternatively, the period Z1 may be set to any desired period.

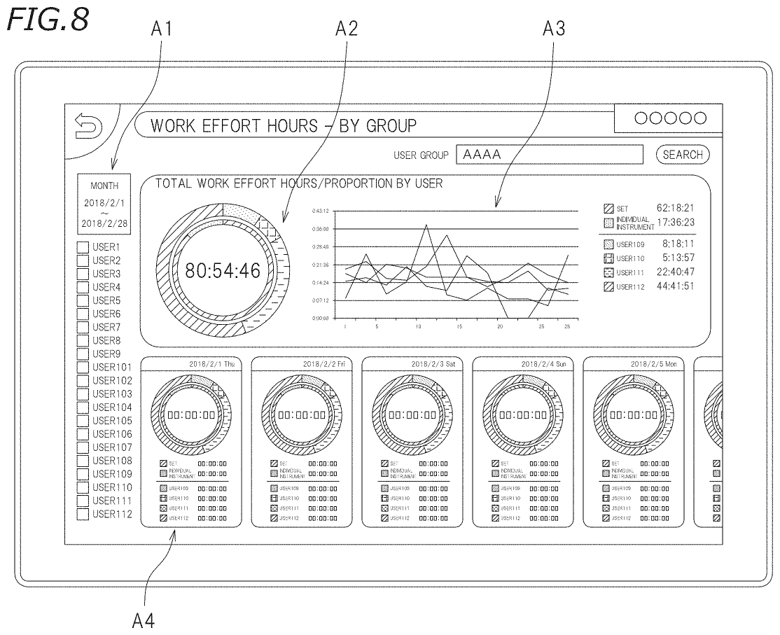

FIG. 8 is a view illustrating a dashboard (information display screen) provided for this system 10.

In this system 10, as illustrated in FIG. 8, the period Z1 is set to a period of from Feb. 1, 2018 to Feb. 28, 2018 in the upper area of the left side A1 of the screen. The dashboard may be configured so that the period Z1 can be changed in that area. FIG. 8 shows that the work information of the users identified by user IDs in a 1 month period of February 2018 is extracted based on the user IDs and the work hours stored in the third memory 103.

In the dashboard shown in FIG. 8, the workers (users) who are registered in the system 10 are listed in the lower area of the left side A1 of the screen. A pie chart A2 at the top left area in the middle of the screen includes a first pie chart, arranged outside, that shows the proportions of the work hours of selected 4 workers (users 109 to 112 in FIG. 8) in a 1 month period of February. A second pie chart arranged inside the first pie chart shows the proportions of work contents (the work proportions for surgical instrument sets and individual instruments) of the selected 4 workers in a 1 month period of February. The inside area of the pie chart A2 shows that the total work hours of the selected 4 workers in a 1 month period of February 2018 is 80:54:46. Each of the line graphs A3 arranged at the top center in the middle of the screen shows the total work hours, day by day, of each of the selected 4 workers in a 1 month period of February 2018. The line graphs A3 may be displayed with different colors respectively for the 4 workers. In addition, the total work hours for each day are aggregated and displayed in the lower area A4 in the middle of the screen. Such items of the information in the dashboard can be obtained by the processes performed by the first processor 201 as described above. It should be noted that FIG. 8 merely shows an example of dashboard, and that the format of the dashboard and the information to be displayed in the dashboard are not limited to those shown in FIG. 8.

The first processor 201 may be configured or programmed to count the work effort hours of users in a predetermined period Z1. In this case, the first processor 201 may be configured or programmed to count the number of work information of a user identified by a user ID in a predetermined period Z1, for example, in a one month period of February 2018.

In this preferred embodiment, as illustrated in FIG. 3, the first memory 101 of the system 10 stores the work unit prices (415, 416) of users in association with the user IDs (411). In this preferred embodiment, the first processor 201 is configured or programmed to calculate the work cost of a user in a predetermined period Z1 based on the work information of the users that is stored in the third memory 103 and the work unit prices of the users that are stored in the first memory 101. In this case, it is possible to calculate, for example, the total work cost of the user in a one month period of February 2018 from the total work hours shown in FIG. 8. The total work cost of the user in the period Z1 may be provided, for example, as an indicator to assess the productivity of the user (worker).

The changes of various types of data that can be extracted by the first processor 201 in a predetermined period Z1 can be expressed by graphs, such as line graphs, as shown in FIG. 8.

The second processor 202 is configured or programmed to extract work information of the work performed on the instruments included in a surgical instrument set identified by a set ID. Herein, as illustrated in FIG. 4, the second processor 202 stores set IDs associated in a one-to-one correspondence with surgical instrument sets, each of which includes a plurality of instruments, and instrument IDs of the instruments included in the surgical instrument sets, so that the set IDs and the instrument IDs are associated with each other. The second processor 202 makes it possible to extract the work information for each of the surgical instrument sets.

The second processor 202 may be configured or programmed to obtain, for example, the work hours of the users for a surgical instrument set identified by a set ID in a predetermined period Z1.

This makes it possible to obtain the work hours of the users required to collect one surgical instrument set in the collection area A2. It is also possible to obtain the work hours of the users required to assemble one surgical instrument set in the assembling area A4.

The second processor 202 may be configured or programmed to calculate the work cost for a surgical instrument set in a predetermined period Z1, based on the work information of the users and the work unit prices of the users for the surgical instrument set. This makes it possible to calculate the work cost (which corresponds to a labor cost) for one surgical instrument set.

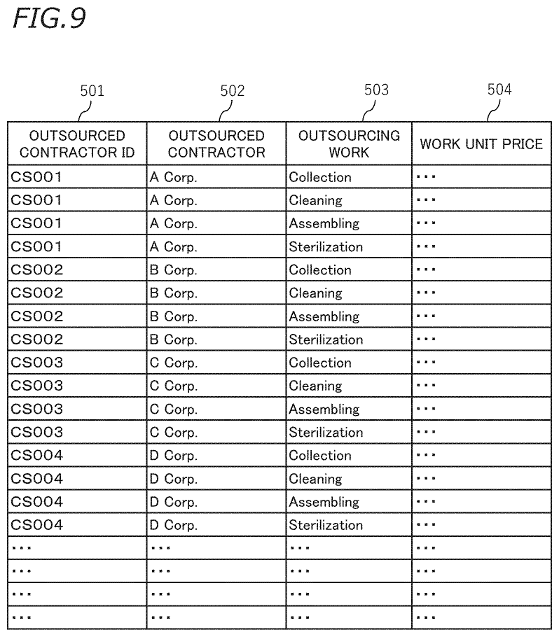

The fifth memory 105 stores work information and outsourcing unit prices in association with each other. FIG. 9 is a view illustrating an example of the configuration of a pre-stored master data table that stores outsourced contractor data, outsourcing work information, and outsourcing unit prices, which are associated with each other. The master data as shown in FIG. 9 may be prepared as the fifth memory 105. In the example shown in FIG. 9, a column 501 to record outsourced contractor IDs, a column 502 to record outsourced contractors, a column 503 to record outsourcing work, and a column 504 to record work unit prices are prepared. In this case, the master data are configured so that the work unit price is identified according to the outsourced contractor and the outsourcing work. It should be noted that the format of the master data corresponding to the fifth memory 105 is not limited to the above-described one, and more detailed conditions may be set for the outsourcing work to be outsourced. It is also possible that the work unit price may be predetermined for each type of the surgical instrument sets to be outsourced.

The fourth processor 204 is configured or programmed to, based on the work information and the outsourcing unit price, obtain the outsourcing cost of the work that is identified by the work information. For example, the work information of a user identified by a user ID in a predetermined period Z1 is extracted by the first processor 201.

The fourth processor 204 may be configured or programmed to, based on the outsourcing unit price and the work information of the user that has been obtained by the first processor 201, calculate the outsourcing cost according to the work information of the user in the predetermined period Z1. Thus, the fourth processor 204 is able to, based on the work information of the user that has been extracted by the first processor 201, calculate the outsourcing cost of the corresponding work when the work is outsourced to an outsourced contractor.

In this case, the fourth processor 204 may further be configured or programmed to, based on the outsourcing unit price and the work information of the work performed for the instruments included in a surgical instrument set, calculate the outsourcing cost for the surgical instrument set in a predetermined period Z1. The outsourcing cost for a surgical instrument set may be calculated based on the outsourcing unit price and the work information of the users for the surgical instrument set in a predetermined period Z1, for example.

Such a process makes it possible to provide the outsourcing cost required in the case of outsourcing the work, which can be compared with the work cost of the users.

In this preferred embodiment, the work information of the work performed on the surgical instruments that is stored in the third memory 103 may include work information classified into the steps of surgical operation, collection, cleaning, assembling, sterilization, storage, and repair, as described earlier. Note that the work information in this preferred embodiment also includes the work information of the surgical operation step and the repair step.

The sixth processor 206 is configured or programmed to extract work performed at a specific work area. Thus, based on the information stored in the third memory 103, the work information can be extracted for each of the steps that are classified as collection, cleaning, assembling, sterilization, storage, and repair. Such a process makes it possible to extract, for example, the amount of work of a user at a specific work area in the period Z1. For example, the amount of work of a user extracted in a predetermined period Z1 at the assembling area A4 may be provided as an indicator to assess the productivity and the proficiency level of the user (worker) in the assembling work.

In this case, the first processor 201 may be configured or programmed to extract the work information of a user identified by a user ID in a predetermined period Z1 for each of the steps. This makes it possible to extract the work information of a specific user in the predetermined period Z1 for each one of the steps. As a result, it is possible to provide an indicator to assess the productivity and the work proficiency level of the user for each one of the steps.

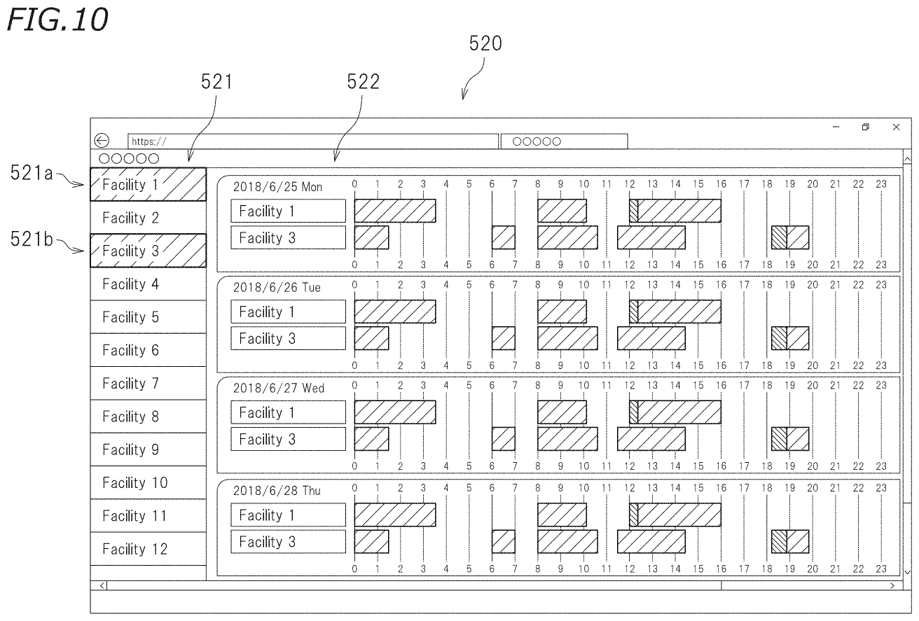

The first processor 201 may be configured or programmed to obtain work effort hours per unit time. In this case, it is possible to obtain data that can assess the intensity level of work per unit time. For example, the work effort hours per unit time may be represented in graphical form. FIG. 10 shows an information display screen 520 is a view illustrating an example of an information display screen containing a graph showing work effort hours per unit time in graphical representation.

In FIG. 10, the registered facilities are listed in a left end column 521. The middle area 522 shows bar graphs colored with different colors according to the work effort hours obtained per unit time for each of the selected facilities (facilities 1 and 3 in FIG. 10) among the registered facilities. Here, items 521a and 521b of the facilities 1 and 3 that are selected in the left end column 521 are colored with different colors on the screen. This allows the user to easily distinguish between the selected facilities 1 and 3. In the middle area 522, bar graphs that are colored with different colors according to the work effort hours for the selected facilities 1 and 3 are shown for each day. For example, the bars may be colored with different colors between a time period in which no work effort hour has been obtained, a time period in which work effort hours have been obtained, and a time period in which the intensity level of work is high. For example, the bar graphs may be colored so that the bars that represent time periods with higher work concentration levels are colored with darker shades of colors. It should be noted that FIG. 10 merely shows a schematic view, in which the bar graphs in the middle area are the same every day, but in reality, the bar graphs actually change depending on the intensity level of work in the facilities 1 and 3.

Here, a predetermined threshold value may be set for the work effort hour, and a time period in which the work effort hour is greater than the predetermined threshold value is considered as a time period in which the intensity level of work is high. The work effort hours per unit time may be represented in various types of graphs, for example, bar graphs or line graphs, based on the work effort hours obtained for each different time period. In addition, although it has been described that the work effort hours are obtained for each different facility, the work effort hours may also be obtained for each different work area. This makes it possible to provide data for finding the time period in which work is intensified, for each of the facilities or each of the work areas. By utilizing the data provided in such a way, the distribution of workers and the number of workers assigned to each time period may be adjusted more appropriately in a department that manages the surgical instruments.

The system 10 herein causes the processor 100 to aggregate information to perform a predetermined process. Each of the control terminals 13a to 13g may be configured or programmed to acquire information from the processor 100 likewise, so as to perform a similar process to the process performed by the processor 100. In other words, each of the control terminals 13a to 13g may be configured or programmed to function as the system 10 on its own. The control terminals 13a to 13g may also be configured or programmed to bidirectionally communicate with the processor 100 to cooperate with the processor 100, and to display required information with the control terminals 13a to 13g, upon receiving the result computed by the processor 100. Each of the control terminals 13a to 13g, serving as a client terminal, may be configured to be a tablet terminal equipped with a camera. In this case, the camera fitted in the tablet terminal may be configured to function as a reading unit of each of the readers 11a to 11g. It is also possible that the screen of the tablet terminal may be configured to function as one of the display devices 12a to 12h.

In addition, the system 10 is able to collectively manage the information about the surgical instrument sets and the instruments that are used in a plurality of hospitals by, for example, gathering the information into the processor 100. In this case, the surgical operation area A1, the collection area A2, and the like are provided in each of the hospitals. In the system 10, the control terminals 13a to 13g in the respective areas A1 to A7 and the processor 100 may be connected to each other so as to allow bidirectional communication with each other.

For example, as illustrated in FIGS. 1 and 2, the external terminal 21 may be connected to the system 10 through the communication network 14. For example, the information that can be provided by the system 10 may be made available to the external terminal 21 connected to the system 10 by using cloud computing technology.

The external terminal 21 may be a terminal that is not installed in the areas A1 to A7. For example, the system 10 may be accessed from a terminal installed in, for example, a department of a hospital that takes charge of administrative work, accounting, inventory control, procurement of instruments and surgical instrument sets, and the like, which does not carry out such work as surgical operation and collection, to acquire the information of the instruments and the surgical instrument sets managed by the system 10. In addition, the external terminal 21 may be a portable terminal that is connectable with the communication network 14, such as a smartphone, a tablet computer, or a laptop computer. In this case, the system 10 is able to be accessed from any desired location to acquire the information of the instruments, the surgical instrument sets, and the users that are managed by the system 10.

The system 10 may incorporate a required authentication process and a required function restriction process to the external terminal 21 as needed. In that case, the system 10 may be configured or programmed to prepare a website that is browsable by a web browser installed in the external terminal 21. The system 10 may be configured or programmed to perform, through the prepared website, an authentication process for the external terminal 21 and provide a predetermined service in response to a request from the external terminal 21, for example.

As described thus far, the system 10 may be configured or programmed to cause the external terminal 21 to display various types of data that can be provided by the system 10 in response to a request from the external terminal 21 connected via the communication network 14. Various types of data that can be provided by the system 10 are not limited to the examples described herein. Examples of the data that can be provided by the system 10 may include information stored in storage devices in the system 10, information computed or extracted through various kinds of processes performed by the system 10, and the graphs and lists created by the system 10.

Hereinabove, various preferred embodiments of the surgical instrument management system proposed in this disclosure have been described, but it should be understood that the surgical instrument management system proposed in this disclosure is not limited to the preferred embodiments described hereinabove. It should be noted that various modifications and alterations of the surgical instrument management systems may be possible. In addition, the features, structures, or steps described herein may be omitted where appropriate, or may be combined in any suitable combinations, unless specifically stated otherwise.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A surgical instrument management system comprising:
a first memory to store user IDs associated in a one-to-one correspondence with users;
a second memory to store instrument IDs associated in a one-to-one correspondence with surgical instruments;
a third memory to store the user IDs, the instrument IDs, work information and work hours of work performed on the instruments each identified by a respective one of the instrument IDs so that the instrument IDs, the work information, the work hours, and the user IDs are associated with each other; and
a first processor configured or programmed to extract work information of one of the users identified by a corresponding one of the user IDs in a predetermined period, based on the user IDs and the work hours stored in the third memory.

2. The surgical instrument management system according to claim 1, wherein the first processor is configured or programmed to obtain total work hours of the one of the users in the predetermined period.

3. The surgical instrument management system according to claim 1, wherein the first processor is configured or programmed to obtain proportions of work hours for each of the users in the predetermined period.

4. The surgical instrument management system according to claim 2, wherein
the first memory is configured to store work unit prices of the users in association with the user IDs; and
the first processor is configured or programmed to obtain a work cost of each of the users based on the work information of the users and the work unit prices of the users.

5. The surgical instrument management system according to claim 1, further comprising:
a fourth memory to store set IDs associated in a one-to-one correspondence with surgical instrument sets, each of the surgical instrument sets including a plurality of instruments; and
a second processor is configured or programmed to extract work information of work performed on the instruments included in the surgical instrument set identified by one of the set IDs.

6. The surgical instrument management system according to claim 5, wherein the second processor is configured or programmed to obtain work hours of the one of the users for the surgical instrument set identified by the one of the set IDs in the predetermined period.

7. The surgical instrument management system according to claim 6, wherein
the first memory is configured to store work unit prices of the users in association with the user IDs; and
the second processor is configured or programmed to obtain a work cost for at least one of the surgical instrument sets in the predetermined period, based on the work information for the surgical instrument sets and the work unit prices of the users.

8. The surgical instrument management system according to claim 1, further comprising a third processor configured or programmed to set the predetermined period.

9. The surgical instrument management system according to claim 1, further comprising:
a fifth memory to store outsourcing unit prices in association with the work information; and
a fourth processor is configured or programmed to, based on the work information and the outsourcing unit prices, obtain an outsourcing cost of work, the work being identified by the work information.

10. The surgical instrument management system according to claim 9, wherein based on the outsourcing unit price and the work information of the one of the users that is obtained by the first processor, the fourth processor is configured or programmed to calculate the outsourcing cost according to the work information of the user in the predetermined period.

11. The surgical instrument management system according to claim 1, wherein
each of the instruments includes item-identifying information; and
the second memory is configured to store the item-identifying information of each of the instruments in association with the instrument IDs; and
the system further comprising:
a reader configured or programmed to read the item-identifying information; and
a fifth processor configured or programmed to, when the reader detects the item-identifying information, identify one of the instrument IDs by the item-identifying information, and store the one of the instrument IDs into the third memory in association with work information and work hours of work performed on one of the instruments that is identified by the one of the instrument IDs and with the user IDs.

12. The surgical instrument management system according to claim 1, wherein
the work information of the work performed on the instruments that is stored in the third memory includes work information classified into the steps of collection, cleaning, assembling, sterilization, and storage; and
the system further comprising a sixth processor configured or programmed to extract work information for each of the steps of collection, cleaning, assembling, sterilization, and storage.

13. The surgical instrument management system according to claim 12, wherein the first processor is configured or programmed to extract work information of the one of the users identified by one of the user IDs for each of the steps.

14. The surgical instrument management system according to claim 1, wherein the first processor is configured or programmed to obtain work effort hours per unit time.
